(19) United States
(12) Patent Application Publication
(10) Pub. No.: US 2020/0271578 A1
(43) Pub. Date: Aug. 27, 2020

(54) TOILET DEVICE

(71) Applicant: LIXIL Corporation, Tokyo (JP)

(72) Inventor: Haruo YAMASAKI, Tokyo (JP)

(73) Assignee: LIXIL Corporation, Tokyo (JP)

(21) Appl. No.: 16/649,619

(22) PCT Filed: May 1, 2018
(86) PCT No.: PCT/JP2018/017412

§ 371 Date: Mar. 20, 2020

(30) Foreign Application Priority Data
Sep. 22, 2017 (JP) 2017-182540
Mar. 28, 2018 (JP) 2018-063358

(51) Int. Cl. G01N 21/552; G01N 33/493; E03D 11/02

(52) U.S. Cl. CPC G01N 21/552; G01N 2201/021; E03D 11/02; G01N 33/493

(57) ABSTRACT

Provided is a toilet device which includes a toilet body including a toilet bowl part, and an analysis device that includes an optical member in contact with urine introduced into the toilet bowl part and that is capable of analyzing a constituent of the urine by detecting analysis light propagated within the optical member and totally reflecting off a urine contact surface of the optical member. An optical path for the analysis light is set such that the analysis light totally reflects off the urine contact surface of the optical member multiple times through multiple reflection. This can increase the number of times the analysis light reflects off the urine contact surface against urine.

### CROSS-REFERENCE TO RELATED APPLICATIONS

This application is a national stage application under 35 USC 371 of International Application No. PCT/JP2018/017412, filed May 1, 2018, which claims the priority of Japanese Application No. 2018-063358, filed Mar. 28, 2018 and Japanese Application No. 2017-182540, filed Sep. 22, 2017, the entire contents of each of which are incorporated herein by reference.

### FIELD OF THE DISCLOSURE

The present invention relates to a toilet device.

### BACKGROUND OF THE DISCLOSURE

There has been conventionally proposed a toilet device in which an analysis device for analyzing urine to identify each urine constituent is built. As an example of such an analysis device, Patent Literature 1 discloses a device for analyzing urine using an ATR (Attenuated Total Reflection) method. In the ATR method, in a state that urine is in contact with a surface (hereinafter referred to “urine contact surface”) of an ATR element as an optical element, an analysis light is totally reflected on the urine contact surface of the ATR element and then detected to obtain a spectrum corresponding to urine composition. Using the spectrum thus obtained, the urine constituent can be analyzed.

Patent Literature 1 Japanese Patent Application Laid-open Publication No. 2009-204598

### SUMMARY OF THE DISCLOSURE

When a small amount of each constituent is to be detected or identified as one of urine constituents, higher detection sensitivity is required for the analysis device. To accomplish the purpose, the inventor has recognized that the number of times the analysis light reflects off the urine contact surface of the optical member is important. Since the technology disclosed in Patent Literature 1 does not disclose anything in this respect, there has been room for improvement.

Embodiments of the present invention have been made in view of such a problem, and a purpose thereof is to provide a toilet device capable of detecting a urine constituent with high sensitivity.

Embodiments of the present invention relate to a toilet device. In some embodiments, the toilet device includes a toilet body including a toilet bowl part, and an analysis device that includes an optical member in contact with urine introduced into the toilet bowl part and that is capable of analyzing a constituent of the urine by detecting analysis light propagated within the optical member and totally reflecting off a urine contact surface of the optical member. An optical path for the analysis light is set such that the analysis light totally reflects off the urine contact surface multiple times through multiple reflection.

In some embodiments, the number of times the analysis light reflects off the urine contact surface against urine can be increased, so that the amount of absorption by the urine in a spectrum corresponding to a specific constituent of the urine can also be increased. As a result, the signal intensity of the absorption spectrum corresponding to the specific constituent is increased, so that the specific constituent can be detected with high sensitivity.

### BRIEF DESCRIPTION OF THE FIGURES

Embodiments will now be described, by way of example only, with reference to the accompanying drawings which are meant to be exemplary, not limiting, and wherein like elements are numbered alike in several Figures, in which:

FIG. 1 is a side view of a toilet device, according to some embodiments;

FIG. 2 is a sectional side view of part of the toilet device, according to some embodiments;

FIG. 3 is a block diagram that shows functions of an analysis device, according to some embodiments;

FIG. 4 is a sectional side view of part of the analysis device, according to some embodiments;

FIG. 5A is a diagram that shows a state where a movable member is placed at a urine sampling position, according to some embodiments;

FIG. 5B is a diagram that shows a state where the movable member is placed at a standby position, according to some embodiments;

FIG. 6A is a diagram of part of a sensor unit viewed from the direction of an arrow A shown in FIG. 4, according to some embodiments;

FIG. 6B is a diagram that shows a urine sample adhered to the sensor unit, according to some embodiments;

FIG. 7 is a flowchart that shows an example of a procedure of processing performed at a control unit and a data processing unit, according to some embodiments;

FIG. 8A is a sectional view taken along line B-B in FIG. 6A, according to some embodiments;

FIG. 8B shows an optical member in a modification, according to some embodiments;

FIG. 8C shows an optical member in another modification, according to some embodiments;

FIG. 9 is a diagram that shows part of a sensor unit, according to some embodiments;

FIG. 10 is a diagram that shows part of a sensor unit, according to some embodiments;

FIG. 11 is a diagram that shows part of a toilet device, according to some embodiments;

FIG. 12A is a diagram that schematically shows part of a toilet device, according to some embodiments;

FIG. 12B is a diagram that shows a state where seal water is discharged, according to some embodiments;

FIG. 13 is a sectional view of part of a sensor unit, according to some embodiments;

FIG. 14 is a sectional view taken along line C-C in FIG. 13, according to some embodiments;

FIG. 15 is a plan view that schematically shows a sensor unit, according to some embodiments;

FIG. 16 is a diagram of the sensor unit viewed from the direction of an arrow D shown in FIG. 15, according to some embodiments;

FIG. 17 is a diagram of the sensor unit viewed from the direction of an arrow E shown in FIG. 15, according to some embodiments;

FIG. 18 is a graph that shows relationships between incident light strength and emitted light strength of analysis light, according to some embodiments;

FIG. 19 is a perspective view that schematically shows an optical member, according to some embodiments;

FIG. 20 is a plan view of a sensor unit, according to some embodiments;

FIG. 21 is a diagram of the sensor unit viewed from the direction of an arrow F shown in FIG. 20, according to some embodiments;

FIG. 22 is a diagram of the optical member viewed from the direction of an arrow G shown in FIG. 20, according to some embodiments; and

FIG. 23A is a schematic diagram of optical paths, according to some embodiments;

FIG. 23B is a schematic diagram of optical paths, according to some embodiments; and

FIG. 23C is a schematic diagram of optical paths, according to some embodiments.

### DETAILED DESCRIPTION OF THE DISCLOSURE

In the following embodiments and modifications, same reference characters denote same constituting elements, and repetitive description will be omitted. Also, in each drawing, part of the constituting elements may be appropriately omitted, or the size of a constituting element may be appropriately enlarged or reduced, for the sake of convenience.

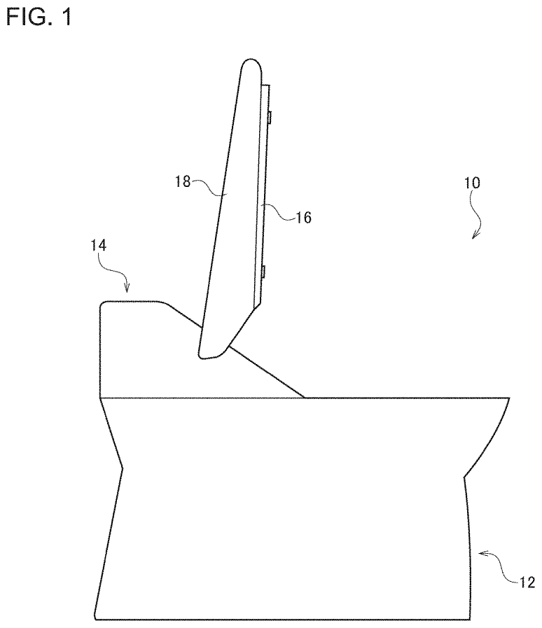

FIG. 1 is a side view of a toilet device 10 according to some embodiments. The toilet device 10 includes a toilet body 12, a toilet seat support member 14, a toilet seat 16, a toilet lid 18, and an analysis device 20 (not illustrated in FIG. 1).

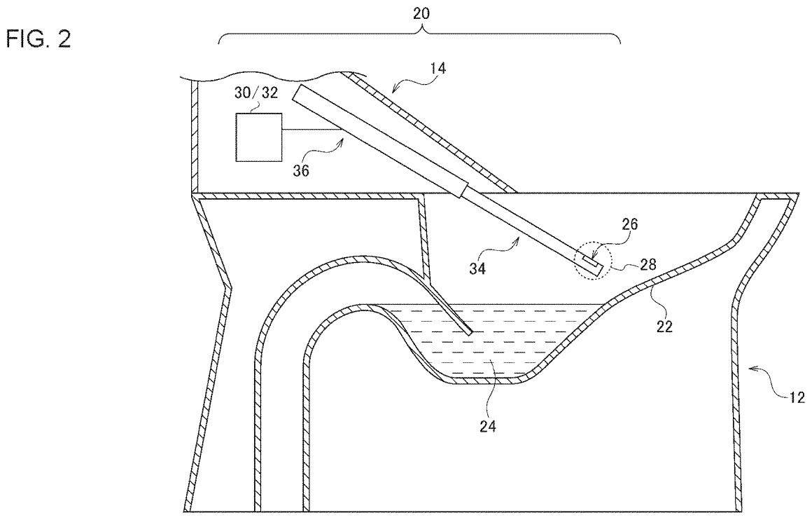

FIG. 2 is a sectional side view of part of the toilet device 10. The toilet body 12 according to some embodiments is a western-style toilet. The toilet body 12 includes a toilet bowl part 22 that receives waste including urine. In a bottom part of the toilet bowl part 22, seal water 24 is stored.

As shown in FIGS. 1 and 2, the toilet seat support member 14 is detachably attached to an upper surface part of a rear part of the toilet body 12 by means of a screw or the like, which is not illustrated. The toilet seat support member 14 has a hollow structure in which a mechanical device, such as a bidet device, is housed. The toilet seat 16 and the toilet lid 18 are attached to the toilet body 12 via the toilet seat support member 14 such as to be openable and closable.

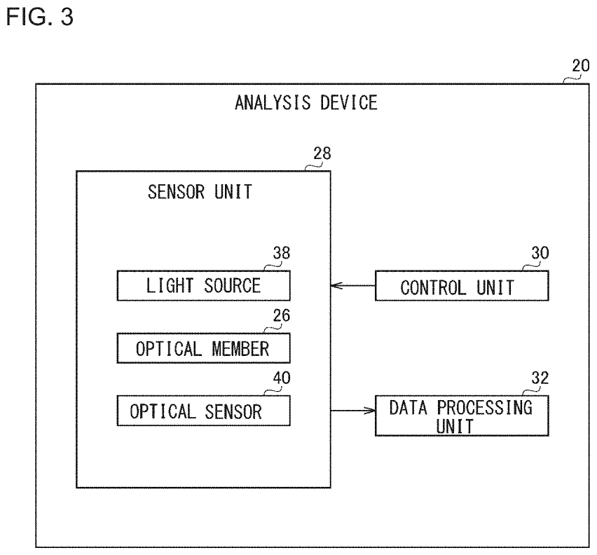

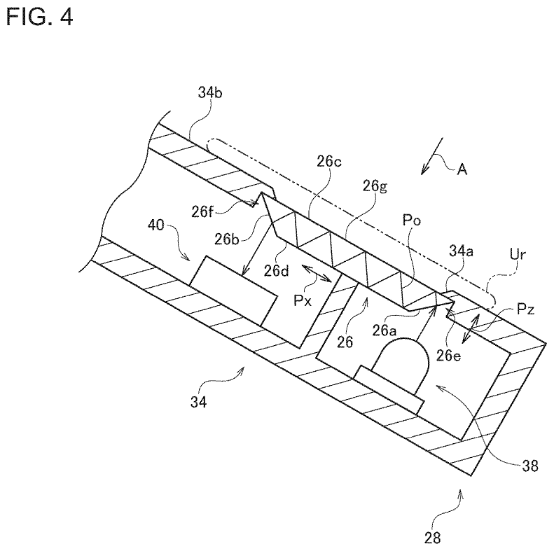

FIG. 3 is a block diagram that shows functions of the analysis device 20 in the toilet device 10. FIG. 4 is a sectional side view of part of the analysis device 20. The analysis device 20 according to some embodiments analyzes the concentration of a urine constituent, for example, using attenuated total reflection as a spectroscopy. The outline of the analysis using attenuated total reflection is as follows. In the attenuated total reflection method, an optical member 26 called an ATR element having a large refractive index is used. The optical member 26 is used in contact with urine Ur to be analyzed (hereinafter, referred to as a urine sample Ur). Into the optical member 26, analysis light is introduced such as to totally reflect off a urine contact surface 26c, which is in contact with the urine sample Ur. By the total reflection of the analysis light, the analysis light partially penetrates as evanescent light into an area on the urine sample Ur side from the interface between the urine contact surface 26c and the urine sample Ur. Since the evanescent light is absorbed at a wavelength specific to the urine sample Ur, by acquiring the spectrum of the analysis light, constituents of the urine sample Ur can be analyzed. The analysis as used herein means qualitative analysis regarding constituents of the urine sample Ur, or quantitative analysis regarding the concentration of the constituent, for example.

The analysis device 20 mainly includes a sensor unit 28, a control unit 30, and a data processing unit 32. Each of the control unit 30 and the data processing unit 32 is implemented by combination of a hardware element and a software element, or only by a hardware element. As a hardware element, a processor, a ROM (read-only memory), or a RAM (random access memory) may be used. As a software element, a program, such as an operating system program and an application program, may be used.

The hardware elements constituting the control unit 30 and the data processing unit 32 according to some embodiments are housed within the toilet seat support member 14. The control unit 30 controls the operation of the sensor unit 28. The data processing unit 32 analyzes constituents of a urine sample based on a detection signal output from an optical sensor 40 (which will be described later) in the sensor unit 28. The processing performed at the control unit 30 and the data processing unit 32 will be described later.

As shown in FIGS. 2 and 4, the sensor unit 28 also includes a movable member 34, a cover member 36, a light source 38, and the optical sensor 40, besides the optical member 26.

The movable member 34 has an elongate shape having a hollow structure. In the movable member 34, the optical member 26, light source 38, and optical sensor 40 are built. These components are housed within the movable member 34 and fixed by means of screws, adhesion, or the like to be built in the movable member 34. The movable member 34 functions as a support member for supporting the components including the optical member 26.

The cover member 36 is disposed within the toilet seat support member 14. The cover member 36 has a cylindrical shape and contains the movable member 34 capable of moving forward and backward. The cover member 36 is detachably attached to the toilet seat support member 14 by means of a screw or the like, which is not illustrated. Accordingly, compared to the case where the sensor unit 28 is directly attached to the toilet body 12, sensor unit 28 can be replaced more easily.

As shown in FIG. 4, the optical member 26 includes a first light incidence surface 26a on which analysis light is incident, a first light emission surface 26b from which analysis light is emitted, and the urine contact surface 26c and a total reflection surface 26d by which the analysis light incident on the first light incidence surface 26a is subjected to multiple reflection to be led to the first light emission surface 26b. The optical member 26 will be detailed later.

The light source 38 can emit analysis light that is incident on the first light incidence surface 26a of the optical member 26. The light source 38 emits, as the analysis light, light within a wavelength range to which infrared light belongs. The wavelength range may be from 1 μm to 15 μm, for example.

The optical sensor 40 can detect the analysis light emitted from the first light emission surface 26b of the optical member 26. The optical sensor 40 may be a pyroelectric sensor, for example. The optical sensor 40 receives analysis light to generate a detection signal corresponding to the analysis light and outputs the detection signal to the data processing unit 32.

In some embodiments, the analysis light emitted from the light source 38 is directly incident on the first light incidence surface 26a of the optical member 26. Being “directly incident” as used herein means being incident without the intervention of any other optical elements, such as a mirror and a prism, in the optical path from the light source 38 to the optical member 26.

Also, in some embodiments, the analysis light emitted from the first light emission surface 26b of the optical member 26 is directly incident on the optical sensor 40. Being “directly incident” as used herein means being incident without the intervention of any other optical elements, such as a mirror and a prism, in the optical path from the optical member 26 to the optical sensor 40.

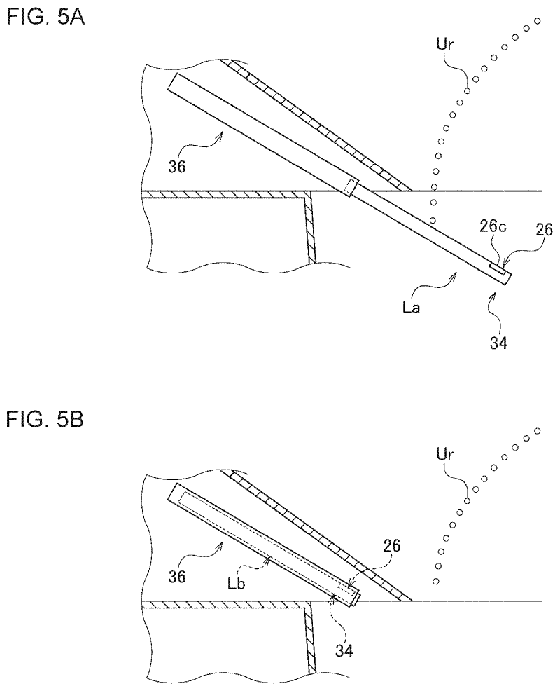

FIG. 5A is a diagram that shows a state where the movable member 34 is placed at a urine sampling position La, and FIG. 5B is a diagram that shows a state where the movable member 34 is placed at a standby position Lb. The movable member 34 is driven to move forward and backward with respect to the cover member 36, by a drive mechanism (not illustrated) configured by combining a motor, a power transmission component, and the like. The movable member 34 according to some embodiments linearly moves forward and backward with respect to the cover member 36, thereby moving between the urine sampling position La and the standby position Lb.

The urine sampling position La is a position where, when the urine sample Ur is introduced into the toilet bowl part 22, the urine sample Ur can be received at the movable member 34. The urine sample Ur may be directly introduced during urination of an examinee, or may be temporarily collected in a container during urination and then introduced from the container. When the movable member 34 according to some embodiments is placed at the urine sampling position La, the movable member 34 is disposed such that the urine contact surface 26c of the optical member 26 faces upward. Also, when the movable member 34 according to some embodiments is placed at the urine sampling position La, the movable member 34 is disposed above the water surface (seal water surface) of the seal water 24 stored in the toilet bowl part 22 (see FIG. 2).

The standby position Lb is a position where, when the urine sample Ur is introduced into the toilet bowl part 22, the urine sample Ur cannot be received at the movable member 34. When the movable member 34 according to some embodiments is placed at the standby position Lb, the entirety of or most of the movable member 34 is housed in the cover member 36, so that urine cannot be received at the movable member 34.

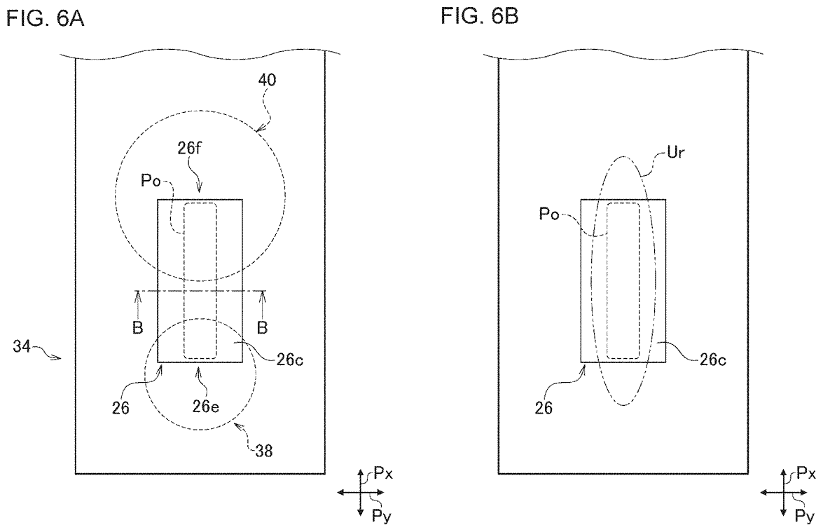

The optical member 26 will now be described. FIG. 6A is a diagram of part of the sensor unit 28 viewed from the direction of an arrow A shown in FIG. 4. As shown in FIGS. 4 and 6, the optical member 26 is a so-called ATR element and formed of a material that transmits the analysis light. The material may be a silicon monocrystal, for example.

The optical member 26 includes the urine contact surface 26c that extends along an extending direction Px. The urine contact surface 26c according to some embodiments extends long along an extending direction Px. Also, the optical member 26 according to some embodiments is an elongate body of plate shape of which the longitudinal direction corresponds to the extending direction Pa.

Each of the first light incidence surface 26a and the first light emission surface 26b is respectively formed in a side edge of the optical member 26 and faces in a thickness direction Pz opposite to the urine contact surface 26c. Each of the first light incidence surface 26a and the first light emission surface 26b is inclined such as to make an obtuse angle with the total reflection surface 26d and to make an acute angle with the urine contact surface 26c.

The first light incidence surface 26a is provided at one end part 26e of the optical member 26 with respect to an extending direction Px, and the first light emission surface 26b is provided at the other end part 26f of the optical member 26 with respect to the extending direction Px. The first light incidence surface 26a is an area where, in an optical path Po for analysis light traveling within the optical member 26, a portion closest to the starting end side is positioned. Also, the first light emission surface 26b is an area where a portion of the optical path Po closest to the termination end side is positioned. The total reflection surface 26d is provided along an extending directions Px of the urine contact surface 26c, on a surface side of the optical member 26 opposite to the urine contact surface 26c.

When the movable member 34 is placed at the urine sampling position La, the urine contact surface 26c is exposed to external space so as to get in contact with the urine sample Ur introduced into the toilet bowl part 22. When the movable member 34 is placed at the urine sampling position La, the entirety of the optical member 26 including the urine contact surface 26c is provided at a position in the air above an inner wall surface of the toilet bowl part 22.

The first light incidence surface 26a faces in one extending direction Px (the lower right in FIG. 4), and the first light emission surface 26b faces in the other extending direction Px (the upper left in FIG. 4). The first light incidence surface 26a is provided such as to extend in the one extending direction Px, from the total reflection surface 26d toward the urine contact surface 26c. Also, the first light emission surface 26b is provided such as to extend in the other extending direction Px, from the total reflection surface 26d toward the urine contact surface 26c.

When the extending directions Px of the urine contact surface 26c are regarded as inclination directions, the urine contact surface 26c includes an inclination region 26g that inclines downward toward one of the inclination directions Px (the lower right in FIG. 4). In some embodiments, the entire urine contact surface 26c corresponds to the inclination region 26g. Hereinafter, directions perpendicular to the inclination directions Px of the urine contact surface 26c and also perpendicular to a normal direction of the urine contact surface 26c will be referred to as width directions Py.

In the movable member 34, a window part 34a is formed such as to penetrate from the outside to the inside of the movable member 34. The optical member 26 is provided such as to cover the window part 34a from the inside of the movable member 34. The urine contact surface 26c of the optical member 26 is provided to be exposed to external space through the window part 34a. An inner peripheral wall surface of the window part 34a is formed such as to extend radially inward, from the outside toward the inside of the movable member 34.

As with the urine contact surface 26c of the optical member 26, an upper surface of the movable member 34 includes an inclination region 34b that inclines downward toward one inclination direction Px of the urine contact surface 26c. The inclination region 34b of the movable member 34 is positioned higher than the inclination region 26g of the urine contact surface 26c and at least provided at a position where the urine sample Ur adhered to the inclination region 34b can be led by its own weight to the urine contact surface 26c. Accordingly, each analysis object person can introduce the urine sample Ur aiming at the inclination region 34b of the movable member 34 besides the urine contact surface 26c of the optical member 26, thereby reducing the work burden on the analysis object person caused by the urine constituent analysis.

As shown in FIGS. 4 and 6, the light source 38 according to some embodiments disposed on one side in an extending direction Px of the urine contact surface 26c. Also, the optical sensor 40 according to some embodiments is disposed on the other side in the extending direction Px. The light source 38 is disposed at a position that overlaps with the one end part 26e of the optical member 26 with respect to an extending direction Px when viewed from a normal direction of the urine contact surface 26c (the viewpoint of FIG. 6). Also, viewed from the same viewpoint, the optical sensor 40 is disposed at a position that overlaps with the other end part 26f of the optical member 26 with respect to the extending direction Px.

Each of FIGS. 4 and 6 schematically shows the optical path Po for analysis light. The optical path Po for analysis light is set such that the analysis light is subjected to multiple reflection on the urine contact surface 26c and the total reflection surface 26d of the optical member 26 to totally reflect off the inclination region 26g of the urine contact surface 26c and the total reflection surface 26d multiple times. The optical path Po for analysis light is also set such that the analysis light is propagated in the inclination direction Px of the inclination region 26g while being totally reflected off the inclination region 26g multiple times. In some embodiments, the optical path Po for analysis light is set such that the analysis light is propagated along the inclination direction Px of the inclination region 26g when viewed from a normal direction of the inclination region 26g (the viewpoint of FIG. 6). In the present specification, “along” includes the case where the subject two directions (the propagation direction in the optical path Po and the inclination direction Px in this example) are nearly identical with each other, besides the case where they are completely identical with each other.

Also, the optical path Po for analysis light forms a strip shape that extends along an inclination direction Px of the inclination region 26g when viewed from a normal direction of the inclination region 26g (the viewpoint of FIG. 6). Further, the width dimension of the optical path Po for analysis light along a width direction Py is set such as to be smaller than the width dimension of the urine contact surface 26c.

Thus, when the optical path Po for analysis light is set, the shape of the analysis light, an incidence angle at which the analysis light is incident on the first light incidence surface 26a, and the shape of the optical member 26 are also set, for example. More specifically, the incidence angle on the first light incidence surface 26a and the shape of the optical member 26, for example, are set such that the optical path Po for the analysis light satisfies the aforementioned conditions between the light source 38 and the optical sensor 40 through the inside of the optical member 26. Particularly, as the shape of the optical member 26, the shapes of the urine contact surface 26c, total reflection surface 26d, first light incidence surface 26a, and first light emission surface 26b of the optical member 26 are set such as to satisfy the conditions. The shape of the analysis light is set such as to satisfy the aforementioned condition regarding the width dimension of the optical path Po for the analysis light. It also can be considered that the sensor unit 28 is configured so that such an optical path Po for the analysis light is set.

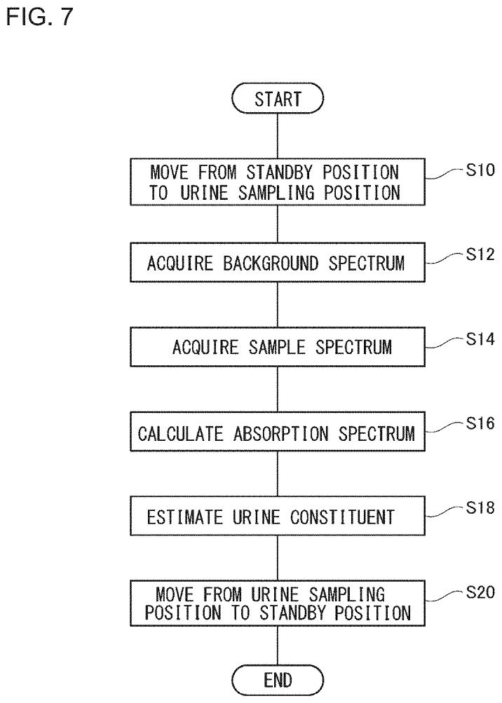

FIG. 7 is a flowchart that shows an example of a procedure of processing performed at the control unit 30 and the data processing unit 32. Upon reception of an analysis start instruction through an operation performed on an operation unit, which is not illustrated, the control unit 30 and the data processing unit 32 perform analysis operation using the analysis device 20. In this analysis operation, the control unit 30 starts with moving the movable member 34 of the sensor unit 28 from the standby position Lb to the urine sampling position La by means of the drive mechanism (S10).

When the movable member 34 is placed at the urine sampling position La, the control unit 30 performs analysis light detecting operation in which the analysis light emitted from the light source 38 is made to pass through the optical member 26 to be detected by the optical sensor 40. In order to acquire a background spectrum as the first stage, the control unit 30 performs the analysis light detecting operation in a state where the urine sample Ur is not in contact with the urine contact surface 26c of the optical member 26. In such a state, the optical sensor 40 detects the analysis light in the analysis light detecting operation, so that the data processing unit 32 acquires a background spectrum (S12).

Thereafter, in order to acquire a sample spectrum as the second stage, the control unit 30 performs the analysis light detecting operation in a state where the urine sample Ur is in contact with the urine contact surface 26c of the optical member 26. As the preceding stage of this operation, the control unit 30 issues a notification for prompting the analysis object person to introduce the urine sample Ur, via a notification unit, such as a speaker. Upon reception of the notification, the analysis object person introduces the urine sample Ur such that the urine sample Ur strikes on the movable member 34. In a state where the urine sample Ur is in contact with the urine contact surface 26c, the optical sensor 40 detects the analysis light in the aforementioned analysis light detecting operation, so that the data processing unit 32 acquires a sample spectrum (S14). The background spectrum or sample spectrum can be acquired by analyzing the frequency of a detection signal output from the optical sensor 40.

The data processing unit 32 calculates an absorption spectrum of the urine sample based on the ratio between the background spectrum and the sample spectrum (S16). The data processing unit 32 then estimates a constituent of the urine sample Ur based on the calculated absorption spectrum (S18). The method of constituent estimation is not particularly limited. For example, the calibration curve method, a chemometric method, or the like may be used for estimation. Since a series of processes in S12, S14, S16, and S18 described above are publicly known, the explanation thereof is simply provided. The data processing unit 32 may output the estimation result regarding a constituent of the urine sample Ur to an output unit, such as a display and a printer.

When the data processing unit 32 completes the estimation of a constituent of the urine sample Ur, the control unit 30 moves the movable member 34 from the urine sampling position La to the standby position Lb by means of the drive mechanism (S20). Thus, the analysis operation using the analysis device 20 is completed.

Through the series of processes, a constituent of the urine sample Ur and the concentration thereof can be analyzed, for example. In this way, the analysis device 20 can analyze a urine constituent by detecting the analysis light totally reflected off the urine contact surface 26c of the optical member 26.

There will now be described the effects of the toilet device 10 according to some embodiments.

(A) The optical path Po for analysis light is set such that the analysis light totally reflects off the urine contact surface 26c of the optical member 26 multiple times. This can increase the number of times the analysis light reflects off the urine contact surface 26c against the urine sample Ur, thereby also increasing the amount of absorption by the urine sample Ur in a spectrum corresponding to a specific constituent of the urine sample Ur. As a result, the signal intensity of the absorption spectrum corresponding to the specific constituent is increased, so that the specific constituent can be detected with high sensitivity. Accordingly, instead of an expensive optical sensor with high sensitivity, a sensor in common use, such as a less expensive pyroelectric sensor with low sensitivity, may be used as the optical sensor 40 to efficiently analyze the urine sample Ur.

The urine contact surface 26c of the optical member 26 includes the inclination region 26g, and the optical path Po for analysis light is set such that the analysis light is propagated in an inclination direction Px of the inclination region 26g. Advantages thereof will be described. The urine sample Ur brought into contact with the optical member 26 flows down in the inclination direction Px by its own weight. Accordingly, as shown in FIG. 6B, the urine sample Ur is spread over a long area, which is elongate in the inclination direction Px, in the inclination region 26g, so that the urine sample Ur covers the long area of the urine contact surface 26c.

The optical path Po for analysis light is set such that the analysis light is propagated in the inclination direction Px of the inclination region 26g. This can increase the number of times the analysis light reflects off the inclination region 26g for the urine sample Ur spread over the long area. As a result, the amount of absorption in a spectrum corresponding to a specific constituent of the urine sample Ur can be further increased, so that the specific constituent can be detected with higher sensitivity. Especially, even when the total volume of the urine sample Ur is small, the urine sample Ur is spread over a long area that is elongate in the inclination direction Px, and the number of times the analysis light reflects against the urine sample Ur can be increased. Therefore, there is the advantage of analyzing a urine constituent with high sensitivity, even when the total volume of the urine sample Ur is small.

When the number of times the analysis light reflects off the urine contact surface 26c is increased in this way, only the dimension in an inclination direction Px of the inclination region 26g needs to be ensured, and the dimension in a width direction Py need not necessarily be ensured. Accordingly, while the number of times the analysis light reflects against the urine sample Ur can be increased, the dimension in a width direction Py of the optical member 26 can be reduced.

As shown in FIG. 5, the movable member 34 is movable between the urine sampling position La at which the urine sample Ur can be received, and the standby position Lb at which the urine sample Ur cannot be received. Accordingly, when the toilet body 12 is used for toilet use without analysis of the urine sample Ur, the movable member 34 will not be impeditive if it is placed at the standby position Lb. Therefore, even if the movable member 34 for receiving the urine sample Ur is built in the toilet device 10, favorable usability can be obtained. In addition, in the case of a failure, a relevant component can be easily replaced.

(B) Since the analysis light emitted from the light source 38 is directly incident on the optical member 26, the propagation efficiency of the analysis light can be improved compared to the case where the analysis light is incident on the optical member 26 via another optical element. Accordingly, the intensity of the analysis light can be ensured, thereby facilitating the detection of a urine constituent with high sensitivity.

(C) Since the analysis light emitted from the optical member 26 is directly incident on the optical sensor 40, the propagation efficiency of the analysis light can be improved compared to the case where the analysis light is incident on the optical sensor 40 via another optical element. Accordingly, the intensity of the analysis light can be ensured, thereby facilitating the detection of a urine constituent with high sensitivity.

Also, for the urine constituent analysis, transportation of urine from the toilet bowl part 22 to the outside is unnecessary. Accordingly, a dedicated analysis device need not be provided outside the toilet body 12, so that a situation where such an analysis device oppressively occupies the toilet space can be avoided. Also, since a transportation mechanism, such as a transportation pump, is unnecessary, costs can be reduced. Also, since the analysis device 20 optically analyzes a urine constituent, the urine constituent analysis can be performed at higher speed, compared to the case where a reagent is used for analysis. Also, compared to the case of using a reagent, less area gets dirty, which facilitates cleaning.

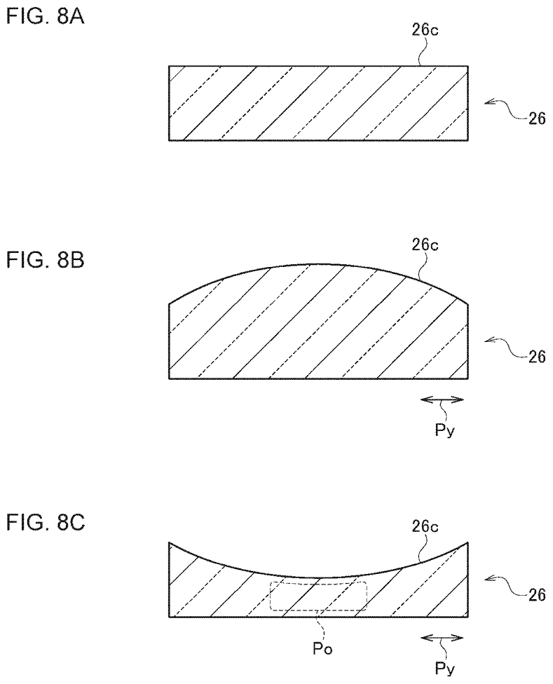

Other features of the toilet device 10 will be described. FIG. 8A is a sectional view taken along line B-B in FIG. 6A. The urine contact surface 26c of the optical member 26 according to the present embodiment is a flat surface that is flat on a cross section perpendicular to an extending direction Px.

Alternatively, the urine contact surface 26c may form an arc shape or a pointed shape with an upward convex on a cross section perpendicular to an extending direction Px, as shown in FIG. 8B. This allows the urine on the urine contact surface 26c to easily flow, by its own weight, in the width directions Py of the urine contact surface 26c, thereby facilitating early removal of the urine from the urine contact surface 26c.

Alternatively, the urine contact surface 26c may form a groove of arc shape or pointed shape with a downward concave on a cross section perpendicular to an extending direction Px, as shown in FIG. 8C. In this case, the optical path Po for analysis light is set such that the analysis light totally reflects off an area corresponding to a groove bottom part of the urine contact surface 26c. Accordingly, the urine sample Ur can be easily collected on the groove bottom part of the urine contact surface 26c, so that, even when the volume of the urine sample Ur is small, a spectrum of the urine sample Ur can be easily obtained.

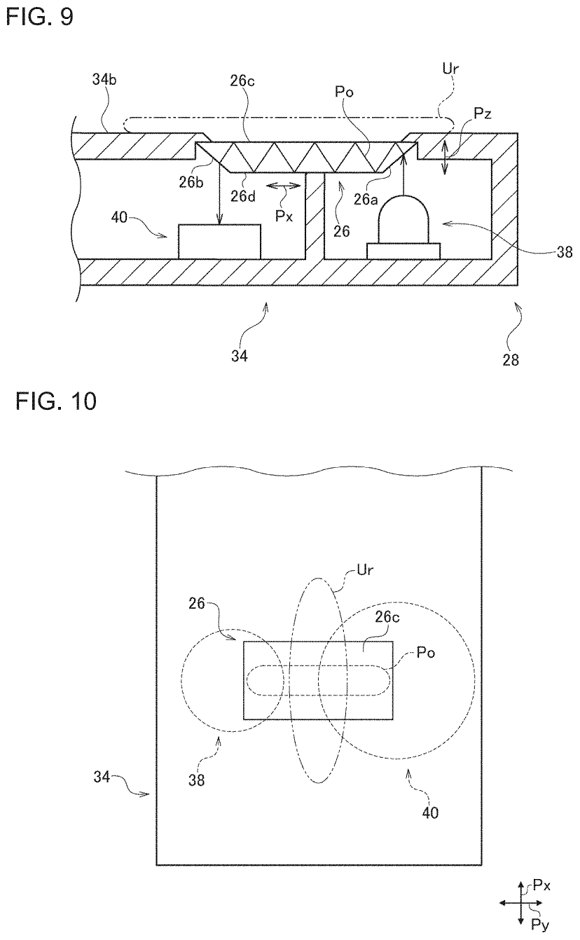

FIG. 9 is a diagram that shows part of the sensor unit 28 in some embodiments. In the example of FIG. 4, an example has been described in which the urine contact surface 26c of the optical member 26 includes the inclination region 26g. Alternatively, the entire urine contact surface 26c may be substantially in parallel with a horizontal plane, as shown in FIG. 9. The same applies to the upper surface part of the movable member 34. Accordingly, since the urine sample Ur remains in contact with the urine contact surface 26c over a long period of time, a constituent of the urine sample Ur can be stably analyzed for a long time.

When the urine contact surface 26c includes the inclination region 26g, at least part of the urine contact surface 26c may be the inclination region 26g. In this case, the propagation direction in the optical path Po for the analysis light has only to include a direction component of an inclination direction Px of the urine contact surface 26c when viewed from a normal direction of the urine contact surface 26c, and the angle between the propagation direction and the inclination direction Px is not particularly limited.

FIG. 10 is a diagram that shows part of the sensor unit 28 in some embodiments. In the example of FIG. 6, an example has been described in which the urine contact surface 26c of the optical member 26 extends long along an inclination direction Px. Alternatively, the urine contact surface 26c may extend long along another direction, as shown in FIG. 10. The urine contact surface 26c of this example extends long along a width direction Py. In this example, the light source 38 is disposed on one side in a width direction Py of the optical member 26, and the optical sensor 40 is disposed on the other side in the width direction Py.

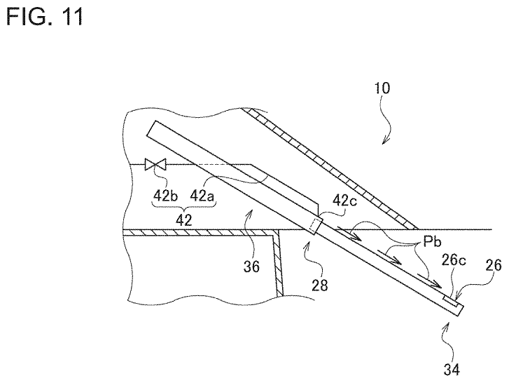

FIG. 11 is a diagram that shows part of the toilet device 10 in some embodiments. The toilet device 10 may include a washing mechanism 42 for washing the urine contact surface 26c. The washing mechanism 42 includes a supply passage 42a through which wash water can be supplied, and an on-off valve 42b with which the supply passage 42a can be opened and closed. The supply passage 42a is provided within the toilet seat support member 14 and used to supply wash water through a supply port 42c, which is provided at a downstream end of the supply passage 42a, to the movable member 34. Through the supply passage 42a according to some embodiments, wash water is supplied to a base end part of the movable member 34 disposed within the cover member 36 and placed at the urine sampling position La. The on-off valve 42b is an electrically driven valve, such as a solenoid valve, and is opened and closed under the control of the control unit 30.

A washing method using the washing mechanism 42 set forth above will now be described. Upon reception of a washing start instruction regarding the optical member 26 through an operation performed on the operation unit, the control unit 30 performs washing operation using the washing mechanism 42. In this washing operation, the control unit 30 starts with moving the movable member 34 of the sensor unit 28 from the standby position Lb to the urine sampling position La by means of the drive mechanism. Thereafter, the control unit 30 opens the on-off valve 42b to supply wash water through the supply port 42c of the supply passage 42a to the movable member 34. When the wash water is supplied to the movable member 34, the wash water flows along the outer surface of the movable member 34 in a direction Pb to be led to the urine contact surface 26c of the optical member 26. The wash water led to the urine contact surface 26c washes off the urine sample Ur, thereby washing the urine contact surface 26c. After the on-off valve 42b is kept open for a predetermined wash water supply time, the control unit 30 closes the on-off valve 42b to stop the wash water supply through the supply passage 42a. Thereafter, the control unit 30 moves the movable member 34 of the sensor unit 28 from the urine sampling position La to the standby position Lb by means of the drive mechanism.

This example has been described as an example in which the washing operation is performed upon reception of the washing start instruction. Alternatively, the washing operation may be performed at timing when the analysis operation is completed. In this case, the operation of supplying wash water to the movable member 34 may be performed between S18 and S20 in FIG. 7.

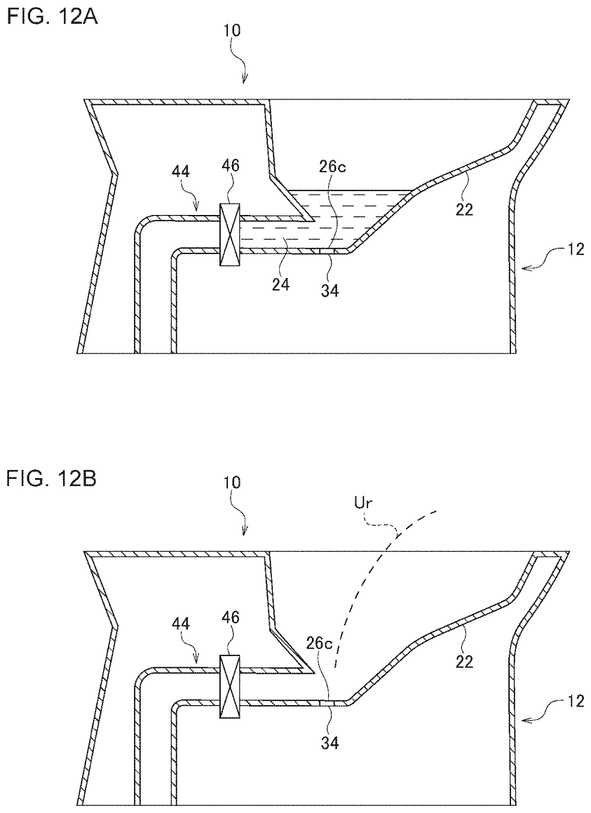

FIG. 12A is a diagram that schematically shows part of the toilet device 10 in the fifth embodiment. The toilet body 12 includes a drainage conduit part 44 connected to a bottom part of the toilet bowl part 22 and functioning as a passage for waste discharged from the toilet bowl part 22 to a sewage line, and an opening/closing structure 46 with which an inner passage of the drainage conduit part 44 can be opened and closed. The opening/closing structure 46 may be a valve structure, such as a flapper valve.

In the example of FIG. 2, an example has been described in which a support member 34, in which the light source 38, optical member 26, and optical sensor 40 are built, is the movable member 34 that is movable with respect to the toilet body 12. The support member 34 in this example is provided to be immovable with respect to the toilet body 12. In FIG. 12A, the light source 38 and the optical sensor 40 are omitted, and only the urine contact surface 26c of the optical member 26 is illustrated. The urine contact surface 26c is provided as part of the inner wall surface of the bottom part of the toilet bowl part 22, at a position to be in contact with urine introduced into the toilet bowl part 22. Alternatively, the urine contact surface 26c may be provided as part of an inner wall surface of the drainage conduit part 44, at a position to be submerged in the seal water 24.

When the toilet device 10 set forth above is used for a purpose other than the analysis of the urine sample Ur, the inner passage of the drainage conduit part 44 is normally closed by means of the opening/closing structure 46 so that the urine contact surface 26c of the optical member 26 is submerged in the seal water 24. This can avoid the situation where the urine sample Ur adhered to the urine contact surface 26c is dried and then firmly adhered to the urine contact surface 26c.

In the analysis of the urine sample Ur, the inner passage of the drainage conduit part 44 is opened by means of the opening/closing structure 46 to discharge the seal water 24 from the toilet bowl part 22, so that the urine contact surface 26c of the optical member 26 is exposed to external space, as shown in FIG. 12B. In this state, the urine sample Ur is brought into contact with the urine contact surface 26c of the optical member 26 so as to be analyzed. When the analysis of the urine sample Ur is completed, the inner passage of the drainage conduit part 44 is closed by means of the opening/closing structure 46, and flush water is supplied into the toilet bowl part 22 by means of a flush water supply mechanism, not illustrated, so that the seal water 24 is stored again.

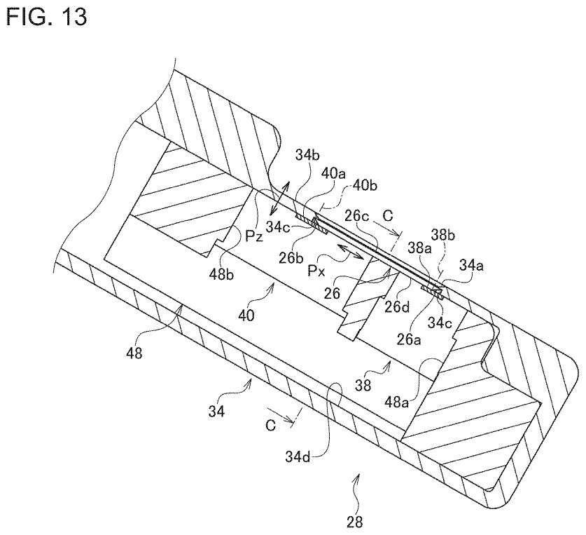

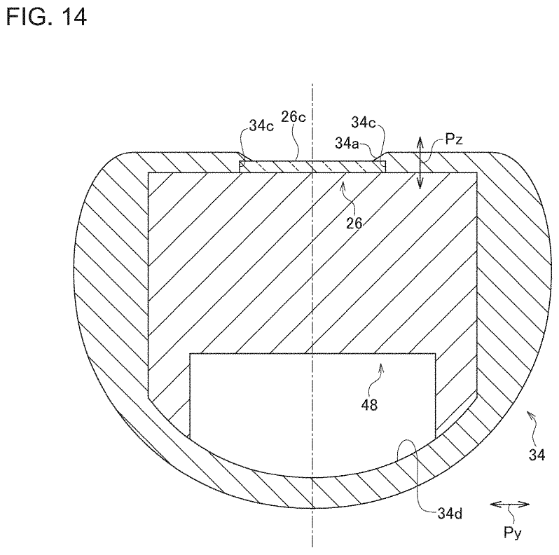

FIG. 13 is a sectional view of part of the sensor unit 28 in some embodiments. FIG. 14 is a sectional view taken along line C-C in FIG. 13. The sensor unit 28 also includes an attachment member 48, besides the optical member 26, movable member 34, cover member 36 (not illustrated), light source 38, and optical sensor 40.

The movable member 34 according to some embodiments includes a positioner part 34c that is in contact with the optical member 26 to position the optical member 26 in the extending directions Px and the width directions Py. The positioner part 34c is provided at a peripheral edge part of the window part 34a in the movable member 34 such as to protrude from an inner wall surface of the movable member 34 toward a thickness direction Pz of the optical member 26. The thickness directions Pz are directions perpendicular to the extending directions Px and the width directions Py of the optical member 26. The optical member 26 is fixed to the movable member 34 by means of adhesion or the like.

The attachment member 48 is shared by the light source 38 and the optical sensor 40, as a member for attachment. More specifically, the attachment member 48 includes a first attached part 48a to which the light source 38 is attached, and a second attached part 48b to which the optical sensor 40 is attached. The first attached part 48a according to some embodiments is a through hole with a step therein, into which the light source 38 can be fitted. In the first attached part 48a, the light source 38 is press-fitted and also caught by the step part, so that the light source 38 is attached to the first attached part 48a. Also, the second attached part 48b according to some embodiments is a through hole with a step therein, into which the optical sensor 40 can be fitted. In the second attached part 48b, the optical sensor 40 is press-fitted and also caught by the step part, so that the optical sensor 40 is attached to the second attached part 48b.

The movable member 34 has an elongate shape having a hollow structure and includes a housing part 34d, which houses the attachment member 48, on one side in a longitudinal direction (the lower right side in FIG. 13). The housing part 34d according to the present embodiment has a shape into which the attachment member 48 can be fitted. The attachment member 48 according to some embodiments can be inserted through an opening part, not illustrated, provided on the other side in the longitudinal direction (the upper left side in FIG. 13) of the movable member 34 and be then moved along the longitudinal direction, so as to be fitted into the housing part 34d. The attachment member 48 is fixed to the movable member 34 by means of screws, adhesion, or the like. The optical member 26 and the attachment member 48 are fixed to the movable member 34 in common.

In the sensor unit 28 described above, the light source 38 and the optical sensor 40 are attached to the attachment member 48 in common. Accordingly, the light source 38 and the optical sensor 40 can be positioned highly accurately, compared to the case where these components are respectively attached to separate members. Since positional deviation due to degradation in positioning accuracy can be prevented, the optical sensor 40 can stably detect the analysis light emitted from the light source 38.

The light source 38 includes a light emitting surface 38a from which analysis light can be emitted, and the optical sensor 40 includes a light receiving surface 40a at which analysis light can be received. The optical sensor 40 receives analysis light at the light receiving surface 40a to detect the analysis light. The light emitting surface 38a and the light receiving surface 40a according to some embodiments are flat surfaces.

When the light emitting surface 38a is projected onto a virtual plane perpendicular to a light emitting axis 38b of the light emitting surface 38a, the projected area is defined as 51. Also, when the light receiving surface 40a is projected onto a virtual plane perpendicular to a light receiving axis 40b of the light receiving surface 40a, the projected area is defined as S2. The light emitting axis 38b is a line extending from the center of the light emitting surface 38a in a direction such that the emission intensity of the analysis light is maximized. Also, the light receiving axis 40b is a line extending from the center of the light receiving surface 40a in a direction such that the light receiving sensitivity of the optical sensor is maximized. In this situation, the area S2 of the light receiving surface 40a is larger than the area 51 of the light emitting surface 38a. Accordingly, even when positional deviation of the optical sensor 40 with respect to the light source 38 occurs, the analysis light emitted from the light emitting surface 38a can be stably received at the light receiving surface 40a.

The light source 38 and the optical sensor 40 may be attached to the first attached part 48a and the second attached part 48b using other means besides fitting. Other means may be screws, adhesion, and the like.

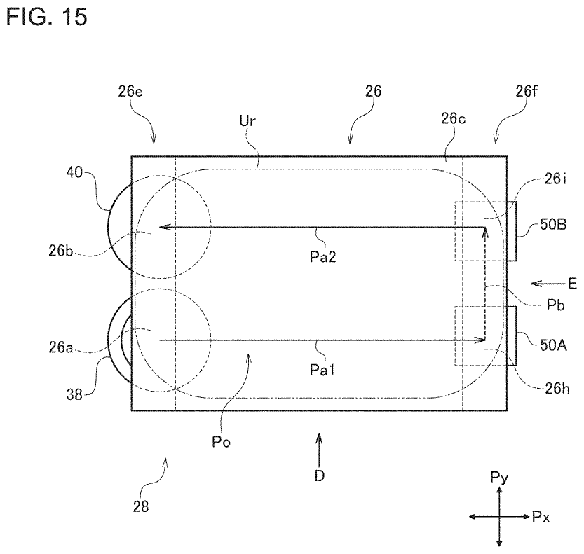

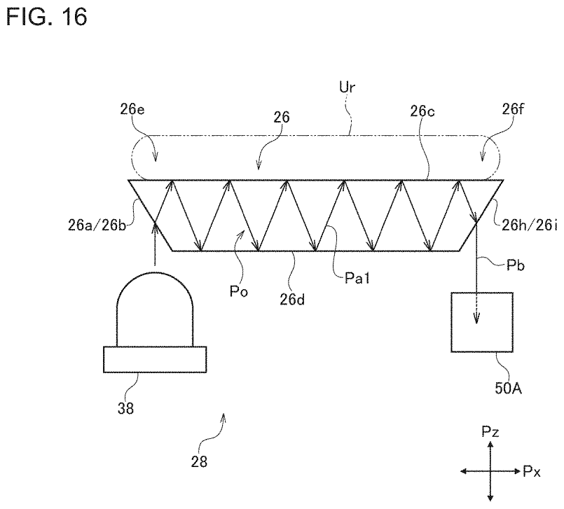

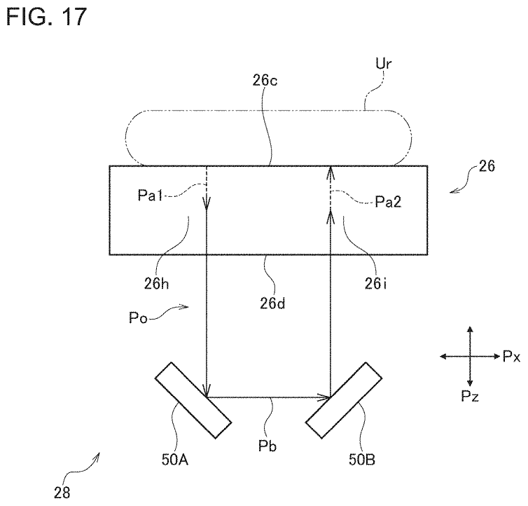

FIG. 15 is a plan view that schematically shows the sensor unit 28 in some embodiments. FIG. 16 is a diagram of the sensor unit 28 viewed from the direction of an arrow D shown in FIG. 15. FIG. 17 is a diagram of the sensor unit 28 viewed from the direction of an arrow E shown in FIG. 15. Although not illustrated, the sensor unit 28 according to some embodiments is used as part of the toilet device 10. In other words, the sensor unit 28 according to some embodiments is built in part of the analysis device 20 set forth above. The sensor unit 28 according to some embodiments includes the optical member 26, the light source 38, the optical sensor 40, and reflecting mirrors 50A and 50B. Although not illustrated, the sensor unit 28 according to some embodiments also includes the movable member 34 in which the optical member 26 is built, and the cover member 36 that contains the movable member 34 capable of moving forward and backward, as described in some embodiments.

The optical member 26 according to some embodiments is an elongate body forming a plate shape, which extends along an extending direction Px (a first direction) and has a thickness in a thickness direction Pz perpendicular to the extending direction Px. The urine contact surface 26c of the optical member 26 is provided on one main surface in a thickness direction Pz, and the total reflection surface 26d thereof is provided on the other main surface. Hereinafter, a direction perpendicular to a thickness direction Pz and an extending direction Px will be referred to as a width direction Py (a second direction). In other words, a width direction Py is an in-plane direction of the urine contact surface 26c perpendicular to an extending direction Px. The in-plane direction as used herein means a direction in parallel with the subject surface.

The optical member 26 according to some embodiments also includes a second light emission surface 26h and a second light incidence surface 26i, besides the aforementioned first light incidence surface 26a, first light emission surface 26b, urine contact surface 26c, and total reflection surface 26d. In some embodiments, each of the light incidence surfaces 26a and 26i and the light emission surfaces 26b and 26h is formed in a side edge part of the optical member 26 and faces in a thickness direction Pz opposite to the urine contact surface 26c. Also, in some embodiments, each of the light incidence surfaces 26a and 26i and the light emission surfaces 26b and 26h is inclined such as to make an obtuse angle with the total reflection surface 26d and to make an acute angle with the urine contact surface 26c.

The first light incidence surface 26a and the first light emission surface 26b are provided in the one end part 26e of the optical member 26 with respect to an extending direction Px. The first light emission surface 26b and the first light incidence surface 26a according to some embodiments are provided in different areas of the same flat surface.

The second light emission surface 26h and the second light incidence surface 26i are provided in the other end part 26f of the optical member 26 with respect to the extending direction Px. The second light incidence surface 26i and the second light emission surface 26h according to some embodiments are provided in different areas of the same flat surface. The second light emission surface 26h is an area where analysis light traveling within the optical member 26 is emitted to the outside, midway along the path from the first light incidence surface 26a to the first light emission surface 26b. The second light incidence surface 26i is an area where the analysis light emitted from the second light emission surface 26h to the outside and then traveling via the reflecting mirrors 50A and 50B is incident.

Viewed from the thickness direction Pz, the light source 38 and the optical sensor 40 according to some embodiments are arranged in a direction Py, at positions that each overlap with the one end part 26e of the optical member 26 with respect to the extending direction Px. In the present embodiment, “viewing from the thickness direction Pz” means the same as viewing from the viewpoint of FIG. 15.

The reflecting mirrors 50A and 50B lead the analysis light emitted from the second light emission surface 26h of the optical member 26 to the second light incidence surface 26i. The reflecting mirrors 50A and 50B according to some embodiments include a first reflecting mirror 50A that reflects the analysis light emitted from the second light emission surface 26h, and a second reflecting mirror 50B that reflects the analysis light reflected by the first reflecting mirror 50A toward the second light incidence surface 26i. Viewed from the thickness direction Pz, the reflecting mirrors 50A and 50B according to some embodiments are arranged in a direction Py, at positions that each overlap with the other end part 26f of the optical member 26 with respect to the extending direction Px.

A path through which the analysis light is subjected to multiple reflection and linearly propagated along an in-plane direction of the urine contact surface 26c within the optical member 26 is defined as a multiple-reflection path. The multiple-reflection path is also regarded as a single path through which the analysis light is subjected to multiple reflection and linearly propagated within the optical member 26, when viewed from the thickness direction Pz.

The optical path Po according to some embodiments includes a plurality of internal optical paths Pa1 and Pa2 that each follow a different multiple-reflection path. This means that, when viewed from the thickness direction Pz, the plurality of internal optical paths Pa1 and Pa2 respectively follow multiple-reflection paths, which have different starting points and different ending points. The plurality of internal optical paths Pa1 and Pa2 constitute part of the optical path Po, which is continuous from the light source 38 to the optical sensor 40. Each of the multiple-reflection paths followed by the plurality of internal optical paths Pa1 and Pa2 is also set such that the analysis light totally reflects off the urine contact surface 26c and the total reflection surface 26d of the optical member 26 to totally reflect off the urine contact surface 26c multiple times.

The plurality of internal optical paths Pa1 and Pa2 according to some embodiments include an outward-side internal optical path Pa1 (a first internal optical path) and a inward-side internal optical path Pa2 (a second internal optical path). The outward-side internal optical path Pa1 follows a multiple-reflection path toward one extending direction Px (the right side in FIG. 15) within the optical member 26, and the inward-side internal optical path Pa2 follows a multiple-reflection path toward the other extending direction Px (the left side in FIG. 15). The outward-side internal optical path Pa1 and the inward-side internal optical path Pa2 constitute part of a path that extends toward one side and turns toward the other side of an extending direction Px. The outward-side internal optical path Pa1 and the inward-side internal optical path Pa2, through which analysis light is propagated, are positioned distantly from each other in the width directions Py. The outward-side internal optical path Pa1 is continuous from the first light incidence surface 26a to the second light emission surface 26h, and the inward-side internal optical path Pa2 is continuous from the second light incidence surface 26i to the first light emission surface 26b.

The outward-side internal optical path Pa1 and the inward-side internal optical path Pa2 are connected with each other via an external optical path Pb that passes outside the optical member 26. The external optical path Pb is continuous from the second light emission surface 26h to the second light incidence surface 26i via the reflecting mirrors 50A and 50B.

(D) The optical path Po for analysis light in some embodiments includes the plurality of internal optical paths Pa1 and Pa2 that each follow a different multiple-reflection path. By increasing the number of the internal optical paths Pa1 and Pa2 for analysis light reflecting off the urine contact surface 26c of the optical member 26, the number of times the analysis light reflects off the urine contact surface 26c against urine can also be increased. This enables detection of a specific constituent of urine with higher sensitivity.

In order to increase the number of times of analysis light reflection using a single internal optical path, elongating the optical member 26 in an extending direction Px may be considered. However, if the optical member 26 is elongated, an area that is not in contact with urine will be likely to occur in the urine contact surface 26c. Accordingly, because of the influence of varied contact areas in contact with urine on the urine contact surface 26c, the number of times the analysis light reflects off the urine contact surface 26c against urine will be varied. Detailed description will be given.

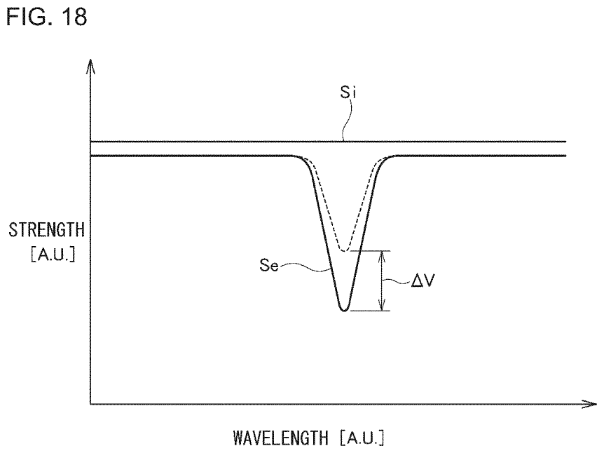

FIG. 18 is a graph that shows relationships between incident light strength Si and emitted light strength Se of analysis light. The incident light as used herein means analysis light emitted from the light source 38 and right before being incident on the optical member 26, and the emitted light as used herein means analysis light emitted from the optical member 26 and right before being incident on the optical sensor 40. The concentration of a urine constituent is estimated based on the difference between the incident light strength Si and the emitted light strength Se. If the number of times the analysis light reflects off the urine contact surface 26c against urine is varied, variance ΔV in emitted light strength Se will occur. Accordingly, variance in difference between the incident light strength Si and the emitted light strength Se will be increased, leading to degradation in accuracy of detecting concentration of a urine constituent. However, in some embodiments, since elongation of the optical member 26 is unnecessary, there is the advantage of detecting a urine constituent with high sensitivity while preventing degradation in detection accuracy due to elongation of the optical member 26.

Also, in order to increase the number of times of analysis light reflection using a single internal optical path, reducing the thickness of the optical member 26 may be considered. In some embodiments, however, since reduction of the thickness of the optical member 26 is unnecessary, there is the advantage of detecting a urine constituent with high sensitivity while ensuring the strength of the optical member 26.

(E) The plurality of internal optical paths Pa1 and Pa2 include the outward-side internal optical path Pa1 and the inward-side internal optical path Pa2 that each follow a multiple-reflection path toward an extending direction Px of the optical member 26. Accordingly, by arranging the internal optical paths Pa1 and Pa2 closer in the width directions Py to each other, even if the positions in contact with urine on the urine contact surface 26c are irregularly located in an extending direction Px, the number of times the analysis light traveling through the internal optical paths Pa1 and Pa2 reflect off the urine contact surface 26c against urine can be ensured more easily. This means that, compared to the case where only a single internal optical path Pa1 or Pa2 is provided, the number of times of reflection on the urine contact surface 26c against urine can be ensured more easily. Therefore, even if the positions in contact with urine on the urine contact surface are irregularly located in an extending direction Px, a urine constituent can be detected with high sensitivity more easily.

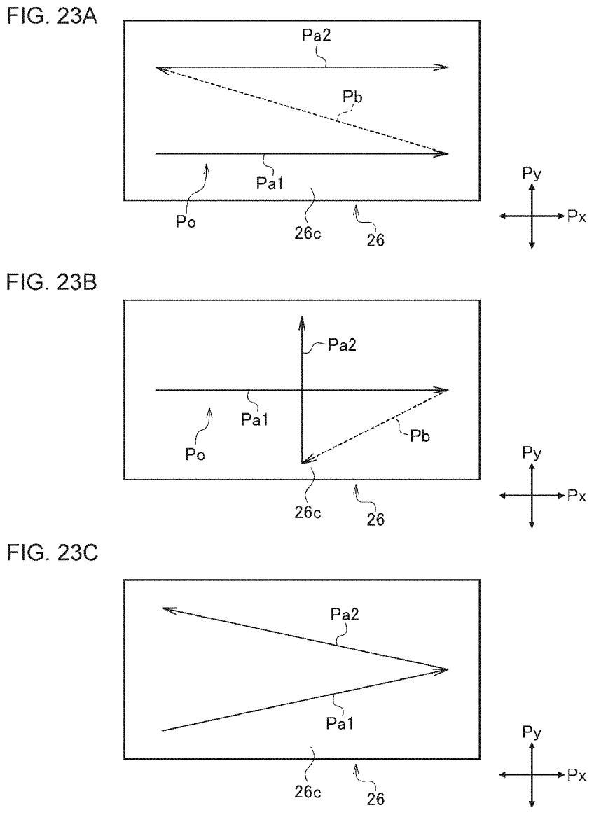

Also, the outward-side internal optical path Pa1 and the inward-side internal optical path Pa2 constitute part of a path that extends toward one side and turns toward the other side of an extending direction Px. Accordingly, compared to the case where two internal optical paths Pa1 and Pa2, through which analysis light is propagated, are provided at the same positions as the outward-side internal optical path Pa1 and the inward-side internal optical path Pa2 and the analysis light is propagated in the same extending direction Px through the two internal optical paths Pa1 and Pa2 (see FIG. 23A), the length of the optical path for the analysis light can be reduced. Therefore, attenuation of analysis light due to increase in length of the optical path for the analysis light can be restrained, so that degradation in detection sensitivity due to such attenuation can be prevented.

Further, with the toilet device 10 employing the sensor unit 28 according to some embodiments, the aforementioned effects (A)-(C) can be obtained.

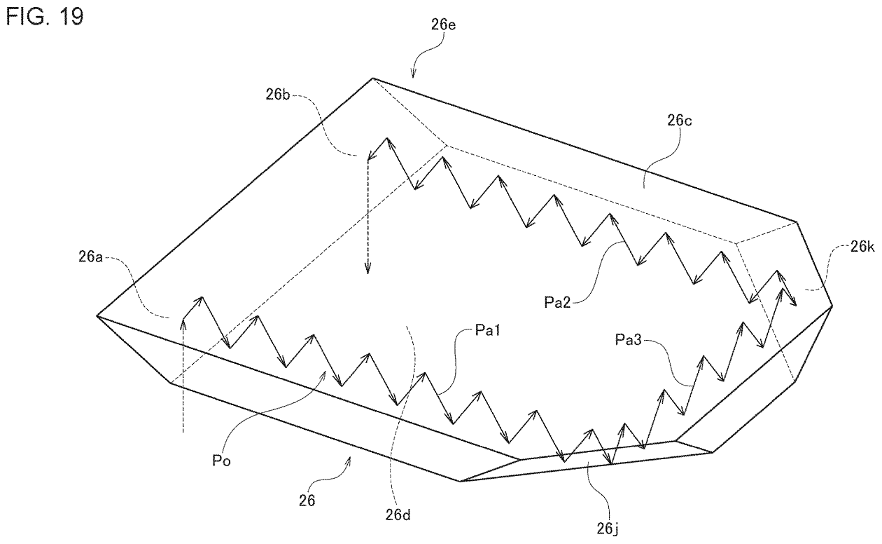

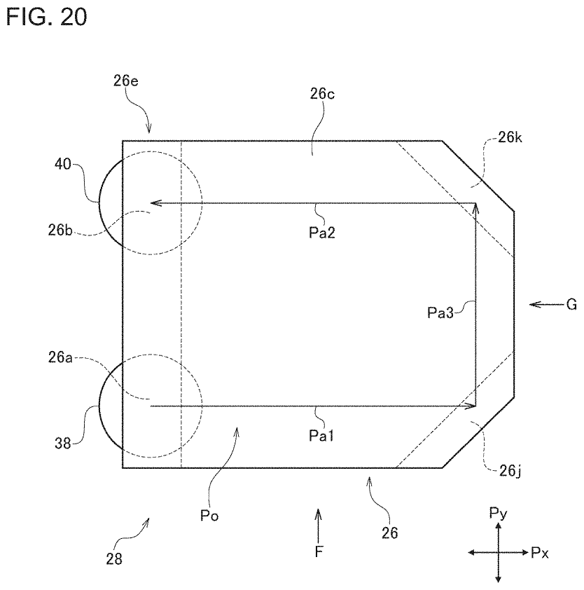

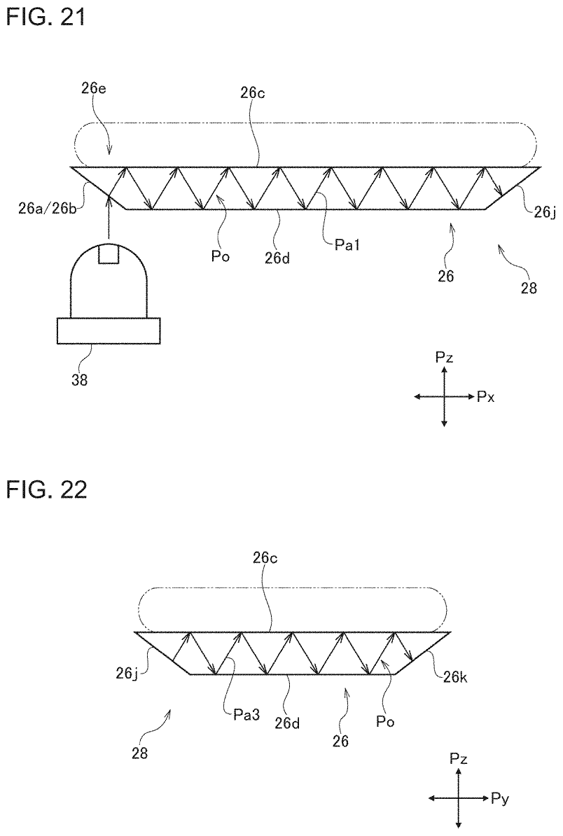

FIG. 19 is a perspective view that schematically shows the optical member 26 in some embodiments. FIG. 20 is a plan view of the sensor unit 28 in some embodiments. FIG. 21 is a diagram of the sensor unit 28 viewed from the direction of an arrow F shown in FIG. 20. FIG. 22 is a diagram of the optical member 26 viewed from the direction of an arrow G shown in FIG. 20. In some embodiments, the sensor unit 28 is also used as part of the toilet device 10. In some embodiments, the sensor unit 28 differs in configuration of the optical member 26. Also, the sensor unit 28 according to some embodiments is not provided with the reflecting mirrors 50A and 50B.

The optical member 26 according to some embodiments also includes a first internal reflection surface 26j and a second internal reflection surface 26k, besides a single first light incidence surface 26a, a single first light emission surface 26b, the urine contact surface 26c, and the total reflection surface 26d as described previously. The first light incidence surface 26a and the first light emission surface 26b are provided in the one end part 26e of the optical member 26 with respect to a longitudinal direction Px. The first internal reflection surface 26j and the second internal reflection surface 26k are provided at the other end side portions of the optical member 26 with respect to the extending direction Px. Each of the first light incidence surface 26a, first light emission surface 26b, first internal reflection surface 26j, and second internal reflection surface 26k is formed in a side edge part of the optical member 26 and faces in a thickness direction Pz opposite to the urine contact surface 26c. Each of these surfaces is inclined such as to make an obtuse angle with the total reflection surface 26d and to make an acute angle with the urine contact surface 26c.

The first internal reflection surface 26j and the second internal reflection surface 26k reflect the analysis light propagated within the optical member 26, toward the inside of the optical member 26. The first internal reflection surface 26j is provided on one side in a width direction Py (the lower side in FIG. 20), and the second internal reflection surface 26k is provided on the other side in the width direction Py (the upper side in FIG. 20). Viewed from the thickness direction Pz, the first internal reflection surface 26j and the second internal reflection surface 26k are inclined with respect to the propagation direction of the analysis light in the outward-side internal optical path Pa1 such as to come closer to each other in the width directions Py toward the propagation direction in the outward-side internal optical path Pa1.

The plurality of internal optical paths Pa1-Pa3 according to some embodiments include, besides the outward-side internal optical path Pa1 and the inward-side internal optical path Pa2, a third internal optical path Pa3 that connects the outward-side internal optical path Pa1 and the inward-side internal optical path Pa2. The third internal optical path Pa3 follows a multiple-reflection path toward a width direction Py within the optical member 26. Each of the internal optical paths Pa1-Pa3 constitutes part of a path that extends toward one side and turns toward the other side of an extending direction Px. Through the outward-side internal optical path Pa1, analysis light is propagated from the first light incidence surface 26a to the first internal reflection surface 26j. Through the third internal optical path Pa3, analysis light is propagated from the first internal reflection surface 26j to the second internal reflection surface 26k. Through the inward-side internal optical path Pa2, analysis light is propagated from the second internal reflection surface 26k to the first light emission surface 26b.

A termination end part of the outward-side internal optical path Pa1 is continuous with a starting end part of the third internal optical path Pa3 at the first internal reflection surface 26j. Also, a termination end part of the third internal optical path Pa3 is continuous with a starting end part of the inward-side internal optical path Pa2 at the second internal reflection surface 26k. This means that the outward-side internal optical path Pa1 and the third internal optical path Pa3 are continuous with each other within the optical member 26, without reflection by a reflecting mirror outside the optical member 26. The same applies to the third internal optical path Pa3 and the inward-side internal optical path Pa2.

(F) Accordingly, when analysis light is to be propagated through two internal optical paths within the optical member 26, reflecting mirrors need not be provided outside the optical member 26. Therefore, the number of parts necessary for the toilet device 10 can be reduced, so that the costs of the toilet device 10 can also be reduced. Further, operational work required to accurately position the reflecting mirrors with respect to the optical member 26 can also be reduced.

Also, the plurality of internal optical paths Pa1-Pa3 are continuous from a single first light incidence surface 26a to a single first light emission surface 26b within the optical member 26. This means that the plurality of internal optical paths Pa1-Pa3 are continuous within the optical member 26, without reflection by reflecting mirrors outside the optical member 26.

(G) Accordingly, when analysis light is to be propagated through the plurality of internal optical paths Pa1-Pa3 within the optical member 26, reflecting mirrors need not be provided outside the optical member 26. Therefore, the number of parts necessary for the toilet device 10 can be further reduced, so that the costs of the toilet device 10 can also be further reduced.

Further, with the toilet device 10 employing the sensor unit 28 according to some embodiments, the aforementioned effects (A)-(E) can be obtained.

Exemplary embodiments and modifications of the present invention have been described in detail. Each of the abovementioned embodiments and modifications merely describes a specific example for carrying out the present invention. The embodiments and modifications are not intended to limit the technical scope of the present invention, and various design modifications, including changes, addition, and deletion of constituting elements, may be made to the embodiments or modifications without departing from the scope of ideas of the invention defined in the claims. In the aforementioned embodiments, matters to which design modifications may be made are emphasized with the expression of “of the embodiment”, “in the embodiment”, or the like, but design modifications may also be made to matters without such expression. Also, the hatching provided on the cross sections in the drawings is not provided to limit the materials of the objects with the hatching.

Although an example has been described in which a western-style toilet is used as the toilet body 12, a Japanese-style toilet, a urinal, or the like may also be used.

The shape of the optical member 26 is not particularly limited as long as it satisfies the condition that analysis light totally reflects off the urine contact surface 26c multiple times through multiple reflection.

As an example of an accessary member used accessorily with and attached to the toilet body 12, the toilet seat support member 14 has been described. The accessary member may be the toilet seat 16 or the toilet lid 18, for example, besides the toilet seat support member 14. The sensor unit 28 may be detachably attached to such an accessary member, as described in some embodiments.

An example has been described in which the movable member 34 linearly moves forward and backward such as to move between the urine sampling position La and the standby position Lb. When the movable member 34 moves between the urine sampling position La and the standby position Lb, a specific moving direction thereof is not particularly limited. For example, the movable member 34 may be supported to be rotatable about a rotational axis with respect to another member, and the movable member 34 may be made movable between the urine sampling position La and the standby position Lb by the rotational operation.

The movable member 34 may be preferably provided separately from a nozzle of a bidet device, which is used to wash the genital area of a human body using washing water, but may be provided integrally with the nozzle.

Although an example has been described in which another optical element is not provided in the optical path from the light source 38 to the optical member 26 or in the optical path from the optical member 26 to the optical sensor 40, such another optical element may be provided.

FIG. 23 are plan views that each schematically show the optical path Po used in the sensor unit 28 in some embodiments. In FIG. 23, only the internal optical paths Pa1, Pa2 and the external optical path Pb of the optical member 26 are shown.

FIG. 23A shows the optical path Po in some embodiments. In some embodiments, the first internal optical path Pa1 and the second internal optical path Pa2 constitute part of a path that extends toward one side and turns toward the other side of an extending direction Px of the optical member 26. The configuration is not limited thereto, and analysis light may be propagated in the same extending direction Px through the first internal optical path Pa1 and the second internal optical path Pa2.

FIG. 23B shows the optical path Po in some embodiments. In some embodiments, through each of the first internal optical path Pa1 and the second internal optical path Pa2, analysis light is propagated in an extending direction Px of the optical member 26. The configuration is not limited thereto, and the analysis light may be propagated along an extending direction Px through the first internal optical path Pa1, and may be propagated along a width direction Py through the second internal optical path Pa2, for example. Also, in this way, analysis light may be propagated through a plurality of internal optical paths that partially overlap with each other.

FIG. 23C shows the optical path Po in some embodiments. The outward-side internal optical path Pa1 and the inward-side internal optical path Pa2 may be continuous with each other at an internal reflection surface (not illustrated) of the optical member 26. Also in this case, the aforementioned effects (D)-(G) can be obtained.

In some embodiments, all the internal optical paths are continuous with each other in the range from the first light incidence surface 26a to the first light emission surface 26b in the optical member 26. Alternatively, at least two of a plurality of internal optical paths may be continuous with each other at an internal reflection surface.

Optional combinations of constituting elements described in the embodiments and modifications are also within the scope of the present invention. For example, the constituting elements in each of the described embodiments may be mutually combined. Also, the inclination region 26g of some embodiments may be provided in the urine contact surface 26c of the optical member 26 in some embodiments. Also, the light source 38 and the optical sensor 40 of some embodiments may be attached to the attachment member 48 in some embodiments. Also, a constituting element may be combined with an arbitrary constituting element in some embodiments.

When the inventions embodied by the embodiments and modifications set forth above are generalized, the following technical ideas are derived.

With regard to the toilet device of some embodiments, when directions in which the urine contact surface extends are regarded as inclination directions, the urine contact surface may include an inclination region that inclines downward toward one of the inclination directions. Also, an optical path for the analysis light may be set such that the analysis light is propagated in one of the inclination directions while totally reflecting off the inclination region a plurality of times. In this mode, urine can be spread over a long area, which is elongate in an inclination direction, in the inclination region of the urine contact surface, and the number of times the analysis light reflects off the urine contact surface against the urine can be increased.

With regard to the toilet device of some embodiments, the analysis device may include a movable member in which the optical member is built, and the movable member may be movable between a urine sampling position at which urine introduced into the toilet bowl part can be received, and a standby position at which urine introduced into the toilet bowl part cannot be received. In some embodiments, when the toilet body is used for toilet use without urine sample analysis, the movable member will not be impeditive if it is placed at the standby position.

With regard to the toilet device of some embodiments, the analysis device may include a light source capable of emitting the analysis light, and the analysis light emitted from the light source may be directly incident on the optical member. In some embodiments, the propagation efficiency of the analysis light can be improved, compared to the case where the analysis light is incident on the optical member via another optical element.

With regard to the toilet device of some embodiments, the analysis device may include an optical sensor capable of detecting the analysis light, and the analysis light emitted from the optical member may be directly incident on the optical sensor. In some embodiments, the propagation efficiency of the analysis light can be improved, compared to the case where the analysis light is incident on the optical sensor via another optical element.

With regard to the toilet device of some embodiments, in any one of the first through fifth modes, the analysis device may include a light source capable of emitting the analysis light, an optical sensor capable of detecting the analysis light, and an attachment member to which each of the light source and the optical sensor is attached. In this mode, the light source and the optical sensor can be positioned highly accurately, compared to the case where these components are respectively attached to separate members. Since positional deviation due to degradation in positioning accuracy can be prevented, the optical sensor can stably detect the analysis light emitted from the light source.

With regard to the toilet device of some embodiments, when a path through which the analysis light is subjected to multiple reflection and linearly propagated along an in-plane direction of the urine contact surface within the optical member is defined as a multiple-reflection path, the optical path may include a plurality of internal optical paths that each follow a different multiple-reflection path. In some embodiments, the number of times the analysis light reflects off the urine contact surface against urine can be further increased, so that a specific constituent of urine can be detected with higher sensitivity.

With regard to the toilet device of some embodiments, the optical member may include a reflection surface that reflects the analysis light within the optical member, and two of the plurality of internal optical paths may be continuous with each other at the reflection surface. In t some embodiments, when analysis light is to be propagated through two internal optical paths within the optical member, reflecting mirrors need not be provided outside the optical member.

With regard to the toilet device of some embodiments, the optical member may include a single light incidence surface on which the analysis light is incident, and a single light emission surface from which the analysis light is emitted. Also, the plurality of internal optical paths may be continuous from the light incidence surface to the light emission surface within the optical member. In some embodiments, when analysis light is to be propagated through a plurality of internal optical paths within the optical member, reflecting mirrors need not be provided outside the optical member.

With regard to the toilet device of some embodiments, the plurality of internal optical paths may include a first internal optical path that follows a multiple-reflection path toward a first direction along an in-plane direction of the urine contact surface, and a second internal optical path that follows a multiple-reflection path toward the first direction. In this mode, by arranging the first internal optical path and the second internal optical path closer to each other in an in-plane direction of the urine contact surface perpendicular to the first direction, even if the positions in contact with urine on the urine contact surface are irregularly located in the first direction, a urine constituent can be detected with high sensitivity more easily.

With regard to the toilet device of an eleventh mode, in the tenth mode, the first internal optical path and the second internal optical path may constitute part of a path that extends toward one side and turns toward the other side of the first direction. In some embodiments, compared to the case where two internal optical paths, through which analysis light is propagated in the same first direction, are provided, the length of the optical path for the analysis light can be reduced.

1. A toilet device, comprising:
a toilet body including a toilet bowl part; and
an analysis device that includes an optical member to be in contact with urine introduced into the toilet bowl part and that is capable of analyzing urine by detecting analysis light propagated within the optical member and totally reflecting off a urine contact surface of the optical member, wherein
an optical path for the analysis light is set such that the analysis light totally reflects off the urine contact surface a plurality of times through multiple reflection.

2. The toilet device of claim 1, wherein:
when directions in which the urine contact surface extends are regarded as inclination directions, the urine contact surface includes an inclination region that inclines downward toward one of the inclination directions; and
an optical path for the analysis light is set such that the analysis light is propagated in one of the inclination directions while totally reflecting off the inclination region a plurality of times.

3. The toilet device of claim 1, wherein:
the analysis device includes a movable member in which the optical member is built; and
the movable member is movable between a urine sampling position at which urine introduced into the toilet bowl part can be received, and a standby position at which urine introduced into the toilet bowl part cannot be received.

4. The toilet device of claim 1, wherein:
the analysis device includes a light source capable of emitting the analysis light; and
the analysis light emitted from the light source is directly incident on the optical member.

5. The toilet device of claim 1, wherein:
the analysis device includes an optical sensor capable of detecting the analysis light; and
the analysis light emitted from the optical member is directly incident on the optical sensor.

6. The toilet device of claim 1, wherein the analysis device comprises:
a light source capable of emitting the analysis light;
an optical sensor capable of detecting the analysis light; and
an attachment member to which each of the light source and the optical sensor is attached.

7. The toilet device claim 1, wherein,
when a path through which the analysis light is subjected to multiple reflection and linearly propagated along an in-plane direction of the urine contact surface within the optical member is defined as a multiple-reflection path,
the optical path includes a plurality of internal optical paths that each follows a different multiple-reflection path.

8. The toilet device of claim 7, wherein:
the optical member includes an internal reflection surface that reflects the analysis light within the optical member; and
two of the plurality of internal optical paths are continuous with each other at the internal reflection surface.

9. The toilet device of claim 7, wherein:
the optical member includes a single light incidence surface on which the analysis light is incident, and a single light emission surface from which the analysis light is emitted; and
the plurality of internal optical paths are continuous from the light incidence surface to the light emission surface within the optical member.

10. The toilet device of claim 7, wherein
the plurality of internal optical paths include
a first internal optical path that follows a multiple-reflection path toward a first direction along an in-plane direction of the urine contact surface, and
a second internal optical path that follows a multiple-reflection path toward the first direction.

11. The toilet device of claim 10, wherein
the first internal optical path and the second internal optical path constitute part of a path that extends toward one side and turns toward the other side of the first direction.
